(19) United States
(12) Patent Application Publication
(10) Pub. No.: US 2014/0093428 A1
(43) Pub. Date: Apr. 3, 2014

(54) DEVICE AND METHOD FOR IMMUNOASSAYS

(71) Applicants: Hélène Briand, l'Arbresle (FR); Bruno Colin, Marcy l'Etoile (FR); Cécile Paris, Bessenay (FR)

(72) Inventors: Hélène Briand, l'Arbresle (FR); Bruno Colin, Marcy l'Etoile (FR); Cécile Paris, Bessenay (FR)

(73) Assignee: BIOMERIEUX S.A., Marcy l'Etoile (FR)

(21) Appl. No.: 14/123,145

(22) PCT Filed: Jun. 4, 2012
(86) PCT No.: PCT/FR2012/051243

§ 371 Date: Nov. 29, 2013

(30) Foreign Application Priority Data
Jun. 16, 2011 (FR) 1155277

(51) Int. Cl. G01N 33/53; B29C 43/46

(52) U.S. Cl. CPC G01N 33/5302; B29C 43/46
USPC 422/69; 264/45.1

(57) ABSTRACT

The present invention relates to a device for performing an immunoassay, the device including a) a support, b) a porous matrix, fixed on the support, the matrix including (i) a liquid sample application area, (ii) a labeling area and (iii) at least one reaction area which includes a test results display area and a verification area, said liquid sample application area labeling area and reaction area being in fluid communication; the device including, at the verification area, at least a part of the porous matrix has pores with dimensions smaller than those of the labeled particles, such that at least a proportion of the labeled particles from the labeling area is stopped at said verification area forming a symbol which is visible or displayable for a user; and a method for manufacturing the device.

The aim of the present invention is a device and a method for performing a test referred to as a lateral flow assay to determine the presence or absence of an analyte in a sample. In particular, the invention relates to new means of verifying the proper operation of the device.

Lateral flow assays, also called quick tests, are commonly used in the fields of clinical, food, pharmaceutical and chemical analyses. Thus, the quick test devices are used to determine the presence of a large number of analytes, such as antibodies, antigens, hormones, proteins, and chemical molecules in liquid samples. These devices generally comprise a support and a matrix which allows the liquid sample to migrate. A distinction is conventionally made between several areas at the matrix which are a liquid sample application area, a labelling area and a reaction area, this latter comprising a capture area and a verification area. These different areas are in fluid communication. Thus, the analyte to be detected, if it is present in the sample deposited at the application area, binds to a first binding partner labelled at the labelling area, the complex thus formed then migrates to the reaction area where it is immobilised at the capture area by reaction with a second binding partner, and the user may determine whether the analyte is actually present by revealing a detectable signal which is determined by the type of label associated with the first binding partner. Generally, the presence of the analyte in the sample is demonstrated in the form of a detectable line, usually referred to as a test line. The reaction also comprises an area for verifying migration of the sample, which will indicate to the user that at least a proportion of the sample has actually passed through the matrix, upstream of the verification area and in particular at the capture area. This may be for example by revealing a verification line of a predetermined colour. By way of example, mention may be made of patent applications WO 2004/003559, WO 2006/092103, WO 2007/081330, and US 2004/0161859. The limits of the verification means currently used in quick tests on strips, integrated or not integrated in a cassette, are that they require additional means at the verification area. This may be, for example, an anti-immunoglobulin immobilised at the verification area which will be capable of capturing and immobilising a labelled antibody or first binding partner. This may also be at the verification zone the addition of a coloured molecule which will be capable of being dissolved and displaced with the sample revealing to the user a colour which is different from that of the coloured molecule after migration of the sample.

The present invention now provides an extremely simple device which does not require additional means in that it uses the labelled particles bound to the first binding partner as a means of revealing and verifying the migration of the sample from sample application area to the reaction area to eliminate the risk of giving false negative results.

The device according to the invention comprises:
a) a support (not shown),
b) a porous matrix (1), fixed on the support, which allows the migration of the liquid sample, said matrix comprising:
(i) a liquid sample application area 2,
(ii) a labelling area 3 comprising at least one first binding partner bound to labelled particles, said first binding partner being capable of binding to said at least one analyte, if it is present in the liquid sample, and
(iii) at least one reaction area 4 comprising:
a test results display area 5 comprising at least one immobilised second binding partner capable of binding to said at least one analyte, and
a verification area 6 which makes it possible to monitor the proper operation of the device downstream of the test results display area 5,
said liquid sample application area 2, labelling area 3 and reaction area 4 being in fluid communication;
said device being characterised in that:
at the verification area 6, at least a part of the porous matrix (1) has pores with dimensions smaller than those of the labelled particles, such that at least a proportion of the labelled particles from the labelling area (3) is stopped at said verification area (6) forming a symbol which is visible or displayable for a user.

The first binding partner and the second binding partner are chosen from the group consisting of antibody, antibody mixture, antibody fragment, mixture of antibody fragments, nanofitin, mixture of nanofitins, antigen, mixture of antigens, protein, mixture of proteins, polypeptide, mixture of polypeptides, peptide, mixture of peptides.

The first binding partner is bound to particles which are labelled by a detectable label, i.e. a compound, a substance which may be detected by visual means, by fluorescent means, by instrumental means, and in particular the labelled particles comprise an elastomer material, preferably a latex, or colloidal gold. However, the particle may also be a magnetic particle.

The proper operation of the device and of sample migration may be displayed by the formation of a symbol for example in the form of a straight line which is substantially perpendicular to the main flow direction of the liquid sample. However, the symbol may also form a trace which admits several distinct tangents, in particular a curvilinear trace, or a broken-line trace, such as a zig-zag.

The verification area 6 comprises a groove arranged in one of the faces of the porous matrix 1, i.e. in the face which is visible to the user or in the face in contact with the support, which makes it possible for the symbol to be formed. The groove may result from the plastic deformation of the porous matrix by crushing of this latter. It is within the general knowledge of the person skilled in the art to determine the useful crushing force which will depend on the nature of the porous membrane, the crushing means and the contact surface of the crushing means against the membrane. The crushing may be performed by means of a device which exerts a localised frontal, continuous or non-continuous, pressure on the membrane or by means of a device which exerts a successive localised, continuous or non-continuous, pressure on the membrane during its movement. Mention may be made, for example, of a blade, a cylinder or a rotating member such as a wheel or a castor. For example, the device which exerts a localised front, continuous or non-continuous pressure, on the membrane may be a cylinder and the pressure exerted by the crushing force may be of between 5 and 50 Newtons, preferably between 10 and 45 Newtons, for example between 10 and 15 Newtons, between 20 and 25 Newtons, between 30 and 35 Newtons or between 40 and 45 Newtons.

Purely by way of illustration, mention may be made of 3 types of nitrocellulose membrane: the Millipore™ HF 135 membrane (reference: HFB 135 UB, batch R9PN61117), the Sartorius™ CN 140 membrane (reference 1UN14ER05002, batch 0509195010900833) and the Sartorius™ CN 150 membrane (reference 1UN15LR05002, batch 07101L3011000933).

Table 1 below gives some examples of pressure which can be applied at various crushing forces onto the above-mentioned nitrocellulose membranes by a cylinder with a diameter of 1.2 mm.

In Table 1, the letter A corresponds to the Millipore HF 135 membrane, the letter B signifies the Sartorius CN 140 membrane and the letter C refers to the Sartorius CN 150 membrane.

TABLE 1

| Test | Membrane | Crushing force (Newtons) |
|---|---|---|
| 1 | A | 10 N-15 N, 20 N-25 N, 30 N-35 N, 40-45 N |
| 2 | A | 10 N-15 N, 20 N-25 N, 30 N-35 N, 40-45 N |
| 3 | B | 10 N-15 N, 20 N-25 N, 30 N-35 N, 40-45 N |
| 4 | B | 10 N-15 N, 20 N-25 N, 30 N-35 N, 40-45 N |
| 5 | C | 10 N-15 N, 20 N-25 N, 30 N-35 N, 40-45 N |
| 6 | C | 10 N-15 N, 20 N-25 N, 30 N-35 N, 40-45 N |

In another embodiment, the groove results from thermal deformation of the structure, for example by means of a laser.

The cross-section of the groove may exhibit the shape of a “U”, a “V” or a rectangle.

The support is made from a liquid-impermeable material, preferably a synthesis synthetic material.

In an embodiment of the device of the invention, a cassette is placed around the matrix and has at least two apertures, a first aperture arranged to allow access to the sample application area 2 and at least a second aperture arranged to allow the user to see the reaction area 4.

The aim of the invention is also a method for manufacturing a device such as described above which comprises the steps of:
producing a device conforming to the state of the art;
making a modification to the matrix, such that the diameter of part of the porous matrix 1 at the verification area 6 is smaller than the diameter of the labelled particles.

The modification of the matrix may be performed by plastic deformation of the matrix by exerting a localised pressure, preferably by rolling at least one rotating member bearing on the external face of the matrix, or by exerting a localised frontal pressure, preferably by applying a pressing member bearing on the external face of the matrix or bearing on the external face of the support.

The rotating member has a rolling diameter of at least 1 mm, preferably a rolling diameter of between 1 and 150 mm and the pressing member has a diameter or an external width preferably of between 0.1 mm and 4 mm, preferably between 0.5 mm and 1.5 mm and the pressure is exerted by a crushing force of between 5 and 50 Newtons, preferably between 10 and 45 Newtons, for example between 10 and 15 Newtons, between 20 and 25 Newtons, between 30 and 35 Newtons or between 40 and 45 Newtons.

### DEFINITIONS

The term “matrix” refers to any type of material which is capable of ensuring the flow and the transfer of a fluid. The fluid can be transferred by capillary force. The matrix may be, for example, made from at least one bibulous material. Bibulous materials are materials which easily absorb a liquid, and across which liquid is transported by capillarity. As non-limiting examples of bibulous materials, mention may be made of nitrocellulose, polyester, fibre-glass, etc.

“Liquid sample” means any sample taken from a patient or individual, and able to contain an analyte such as defined below. This sample may in particular be a liquid biological sample, such as a sample of blood, serum, plasma, saliva, urine, cerebrospinal fluid, pleural fluid, or peritoneal fluid. However the biological sample also comprises semi-solid or solid samples insofar as they can be transformed into a liquid sample by any appropriate method, for example a food specimen, a stool specimen, a tissue specimen, cell culture specimens, or a mucus specimen. This biological sample is obtained through any sampling known to the person skilled in the art. The sample may also be a sample of environmental origin, i.e. a liquid, solid or semi-solid sample from the environment, such as effluents, muds, soils, plants etc. Of course, when the sample is solid or semi-solid, it must be pre-treated to be transformed into a liquid sample.

“Analyte” primarily means an antigen, an antibody, a hormone, a protein or a chemical molecule.

When the analyte is a protein or an antigen it can be detected by binding partners, for example receptors, antibodies, antibody fragments, nanofitins™ and any other ligand capable of binding to a protein or to an antigen.

The binding partner antibodies are for example either polyclonal antibodies, or monoclonal antibodies.

Polyclonal antibodies can be obtained by immunisation of an animal with the appropriate immunogen, followed by the recovery of the antibodies sought in purified form, by sampling the serum of said animal, and separation of said antibodies from the other constituents of the serum, in particular by affinity chromatography on a column on which is fixed an antigen specifically recognised by the antibodies.

Monoclonal antibodies can be obtained by the hybridoma technique, the general principle of which is reiterated below.

In a first stage, an animal, generally a mouse, is immunised with the appropriate immunogen, the B lymphocytes of which are then capable of producing antibodies against this antigen. These antibody-producing lymphocytes are then fused with “immortal” myeloma cells (murine in the example) to give rise to hybridomas. From the heterogeneous mixture of cells thus obtained, cells are then selected that are capable of producing a particular antibody and of reproducing indefinitely. Each hybridoma is reproduced in the form of a clone, each leading to the production of a monoclonal antibody, whose recognition properties with respect to the protein will be testable for example by ELISA, by immunotransfer (Western blot) in one or two dimensions, by immunofluorescence, or by means of a biosensor. The monoclonal antibodies thus selected are then purified, notably in accordance with the affinity chromatography technique described above.

The monoclonal antibodies may also be recombinant antibodies obtained by genetic engineering, by techniques well known to the person skilled in the art.

The nanofitins (trade name) are small proteins which, like antibodies, are capable of binding to a biological target thus making it possible to detect, capture or simply target it within an organism.

The specific binding partners of the protein or the antigen sought in the method of the invention can be used as a capture reagent, a detection reagent or capture and detection reagents.

When the analyte is an antibody it can be detected by binding partners, for example proteins, peptides, anti-immunoglobulins and any other ligand capable of binding to the antibody.

The specific binding partners of the antibody sought in the method of the invention can be used as a capture reagent, a detection reagent or as capture and detection reagents.

The immunological reactions, i.e. of the protein/binding partner binding, antigen/binding partner binding, antibody/binding partner binding can be displayed by any detection means by means of a label, of the binding partner.

Thus, the binding partner can be bound to labelled particles, capable of generating a detectable signal, i.e. comprising a compound, a substance which can be detected by visual, fluorescent or instrumental means.

A non-limiting list of these labelling reagents consists in:
metallic or alloy particles, such as colloidal gold particles,
polymer particles, such as coloured latex particles,
magnetic particles,
fluorescent molecules,
chemiluminescent molecules.

In the embodiments of the invention, the signal generated at the results display area and the signal generated at the positive verification area will, preferably, be of the same kind and will exhibit the same colours.

By way of an example of immunological tests such as defined above, mention may be made of the “sandwich” and “competition” methods.

### FIGURES

FIG. 1:

FIG. 1A is a top view of an embodiment of the device of the invention, before application of the sample. The device of the invention comprises a support (not shown), a matrix 1 comprising a sample application area 2, a labelling area 3, a reaction area 4 comprising a test results display area 5 comprising means for displaying the test results and a sample migration verification area 6. Optionally, the matrix 1 may also comprise a sample absorption area 7.

FIG. 1B shows the results obtained with the device of FIG. 1A after application of a sample which is negative for an analyte to be determined. As shown in FIG. 1B, a signal materialises in the verification area 6, in the form of a straight line perpendicular to the main flow direction of the sample.

FIG. 1C shows the results obtained with the device of FIG. 1A after application of a sample which is positive for an analyte to be determined. As shown in FIG. 10, two signals have materialised, one in the results display area 5 and the other in the verification area 6, each in the form of a straight line perpendicular to the main flow direction of the sample.

FIG. 2:

FIG. 2 is a profile view of two embodiments of the device shown in FIG. 1A.

FIG. 2A shows an embodiment of the matrix 1 which is made up of at least 3 membranes of bibulous materials, identical or different, in fluid communication with one another. Preferably, the 3 membranes are made from different materials, for example one fibre-glass membrane which constitutes the liquid sample application area 2, a polyester membrane which constitutes the labelling area 3 and a nitrocellulose membrane which constitutes the reaction area 4. Moreover, the matrix may comprise a 4th membrane made of an absorbent material which constitutes a sample absorptivity area, such as shown at 7 in this figure.

FIG. 2B shows another embodiment of matrix 1 which is constituted of a single membrane made of a bibulous material on which the sample application area 2, the labelling area 3, the reaction area 4, and potentially the absorptivity area 7, are defined.

FIG. 3:

FIG. 3A shows a pressing member 8 for applying a crushing force F onto the matrix 1. The force F is applied uniformly and perpendicular to the surface of the matrix 1. In this non-limiting embodiment, the pressing member 8 comes into contact with the matrix 1 at a determined force F, causing the deformation of the matrix. The pressing member 8 has a contact surface with the matrix 1 having a width L and a length at least equal to the width of the matrix 1. The pressure is a localised and frontal pressure.

FIG. 3B shows a pressing member 9 for applying a crushing force F onto the matrix 1. The force F is applied uniformly and perpendicular to the surface of the matrix 1. In this non-limiting embodiment, the pressing member 9 comes into contact with the matrix 1 at a determined force F, causing the deformation of the matrix. The pressing member 9 has a radius R and a length at least equal to the width of the matrix 1. The pressure is a localised and frontal pressure.

FIG. 3C shows a rotating member 10 for applying a crushing force F onto the matrix 1. The force F is applied progressively, perpendicular to the surface of the matrix 1, by movement of the rotating member 10 on the matrix. The pressure is a localised and successive pressure.

### EMBODIMENTS

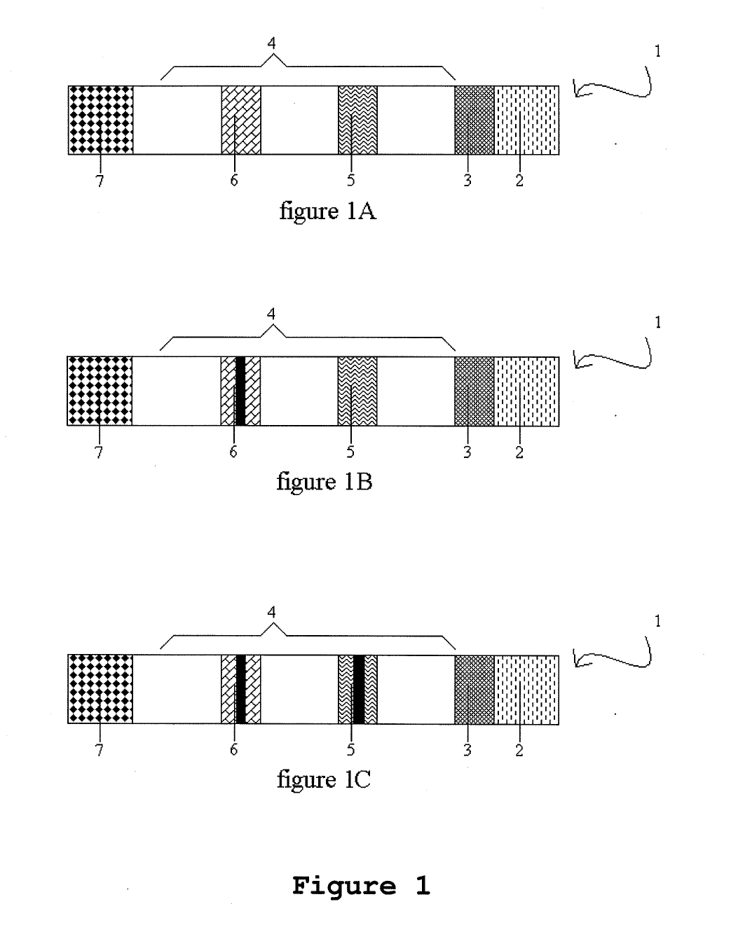

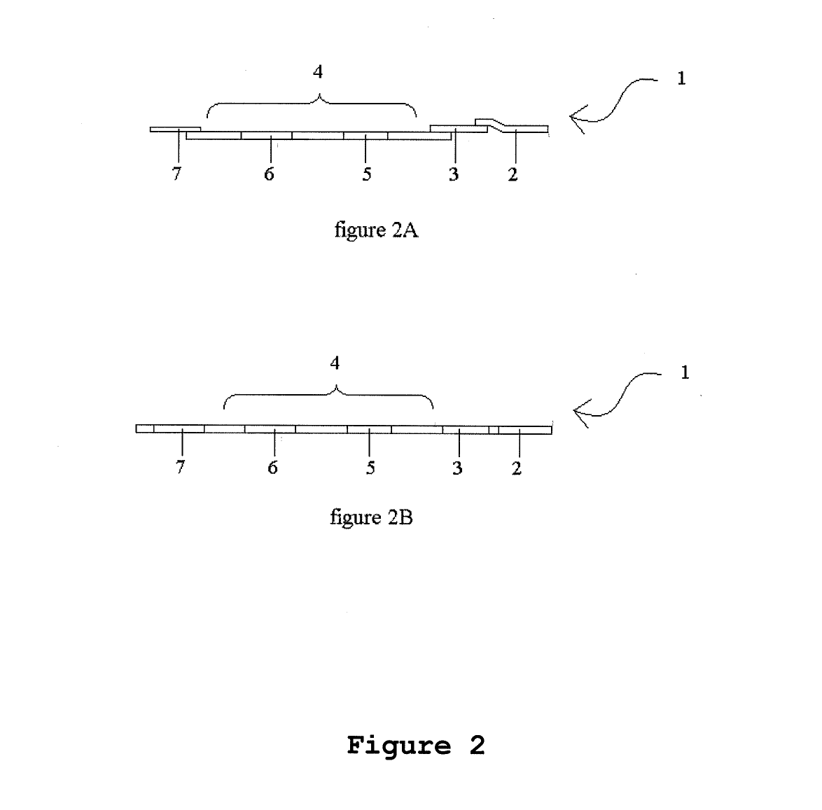

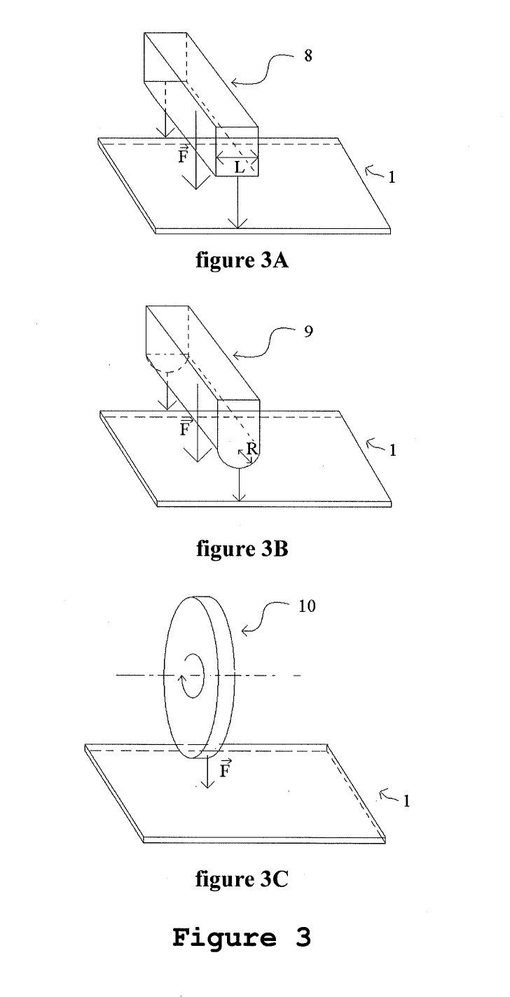

In one embodiment, with reference to FIG. 1, the porous matrix 1 is shown in the form of a rectangular strip, the longitudinal axis of which is in the horizontal position. Areas 2, 3 and 4 are in fluid communication. Area 3 comprises the first binding partner bound to labelled particles, for example an antibody bound to particles bearing a visible or displayable label, such as coloured latex particles, colloid gold particles, etc. This reagent can migrate freely across the matrix in the presence of the liquid sample deposited in area 2 and react with the analyte (antigen) to be determined if it is present. In area 5 of the matrix 1, the second binding partner, for example an antibody having a specificity for an epitope of the antigen which is different from that recognised by the first labelled antibody, is immobilised. In area 6 of the porous matrix 1, a modification is made such that the dimensions of the pores are smaller than those of the labelled particles in order to be able to stop said labelled particles and form a symbol which is displayed or displayable by the user after the fluid flow has passed. FIGS. 1B and 10 illustrate the operation of the test in the presence of a negative control and positive samples.

As shown in FIG. 1B, the sample being a negative control, there is no emission of a signal detectable in the results display area 5. On the contrary, there is formation of a detectable symbol in the migration verification area 6, in the form of, for example, a line perpendicular to the direction of the flow of the liquid sample, which signifies on the one hand that the negative control sample has indeed migrated to area 6 and on the other hand that the device is operational.

As shown in FIG. 10, the sample being positive, there is emission of a signal detectable in the results display area 5 in the form of a line perpendicular to the direction of flow of the liquid sample. There is also formation of a detectable symbol in the migration verification area 6, in the form of, for example, a line perpendicular to the direction of the flow of the liquid sample, which signifies on the one hand that the positive sample has indeed migrated to area 6 and on the other hand that the device is operational.

### EXAMPLES

### Example 1

### Assaying of Anti-HIV-1 Group M, Anti-HIV-1 Group O and Anti-HIV-2 Antibodies

The assay of anti-HIV-1 and anti-HIV-2 antibodies by the test shown in FIG. 1 consists in a sandwich immunological reaction in a step based on an immunochromatographic technique.

Blue latex particles marketed by the company VARIAN (trade name) are coated with a mixture of three peptides specific to the HIV-1 group M, HIV-1 group O and HIV-2 viruses, respectively. These particles are then distributed over a polyester membrane (Ahlstrom-trade name). The membrane is dried for one night at 37° C.

Capture peptides identical to the peptides described above are coated by distribution with a BIODOT (trade name) apparatus onto 3 different nitrocellulose membranes: a Millipore HF 135 unsupported membrane (reference HFB 135UB, batch No. R9PN61117, membrane A), a Sartorius CN 140 supported membrane (reference 1UN14ER050020, batch No. 0509195010900833, membrane B) and a laminated Sartorius CN 150 membrane (reference 1UN15LR050025, batch No. 07101L3011000933, membrane C) in the test results display area 5. The distribution is performed by the same BIODOT apparatus.

The positive control of the test is constituted by crushing the nitrocellulose membrane at the verification area 6. After distribution of the capture peptides, each membrane is dried for one night at 37° C.

After drying, the two membranes, polyester and nitrocellulose, are assembled on a fast test support (backing, by the company G&L (trade name) in association with a sample pad (fibre-glass membrane which acts as a filter for the sample with contact with the particles) and an absorbent pad (an absorbent which has the ability to adsorb the rest of the sample after migration along the different types of membrane). It is mounted in the cassettes after cutting into strip form.

The tested samples are well-characterised HIV-positive samples. The negative samples tested correspond to a pool of negative serum originating from the “Etablissement Français du Sang” (EFS) of the Rhône-Alpes region.

The reading is performed visually with the aid of a reading card which allows signal intensities to be attributed in accordance with the intensity of the blue colour observed.

This card is graduated from L1 to L10. A sample is considered positive if a blue colour appears with an intensity corresponding to at least L4 on the reading scale.

The verification lines were prepared by crushing each membrane at different crushing force in the verification area 6, as indicated in table 2 below, using a 1.2 mm cylinder.

The results are presented in Table 2 below:

TABLE 2

| | | Verification | | Verification |
|---|---|---|---|---|
| | Test line | line | Test line | line |
| Membrane A | Crushing at 20 Newtons | | Crushing at 31 Newtons | |
| Negative | L1 | L7 | L1 | L8 |
| Positive | L10 | L7 | L10 | L7 |
| Membrane B | Crushing at 30 Newtons | | Crushing at 44 Newtons* | |
| | | | Crushing at 40 Newtons** | |
| Negative | L1 | L8 | L1 | L8 |
| Positive | L10 | L8 | L10 | L8 |
| Membrane C | Crushing at 30 Newtons | | Crushing at 41 Newtons* | |
| | | | Crushing at 43 Newtons** | |
| Negative | L1 | L8 | L1 | L8 |
| Positive | L10 | L8 | L10 | L8 |

*negative sample
**positive sample

The results show that in the case of a negative sample, only the positive control is detected at the verification line: a strong colour intensity (L7,L8) regardless of which type of membrane is used. These results make it possible to confirm that the absence of a signal at the test line 5 is due to the negativity of the sample and not to a functional fault of the cassette used for the assay. In the case of a positive sample, the test line and the verification line are displayed with a strong colour intensity, which signifies that the antibodies present in the sample have been captured by the peptides bound to blue latex particles (functionalised latex particles) to form a labelled complex which was then immobilised with the capture peptides at the test line 5 and that the excess of functionalised latex particles was stopped at the verification area 6, confirming the functionality of the cassette.

### Example 2

### Preparation of the Verification Line by Crushing on the Support

The device was prepared in accordance with the protocol described in example 1. The only difference is that the verification lines were prepared by crushing the support at a crushing force of 40 Newtons at the verification area 6, using a 0.6 mm cylinder. The crushing was performed after assembly of the membranes and the support in the external face of the support, causing deformation of the membrane on its face in contact with the support.

A negative sample corresponds to a pool of negative serum originating from the “Etablissement Français du Sang” (EFS) of the Rhône-Alpes region was passed over each membrane to validate this embodiment of the invention.

The reading is performed visually with the aid of a reading card which allows signal intensities to be attributed in accordance with the intensity of the blue colour observed as described in example 1. The results are presented in Table 3 below.

TABLE 3

| | | |
|---|---|---|
| Membrane A | Test line | Verification line |
| Negative | L1 | L8 |
| Membrane B | Test line | Verification line |
| Negative | L1 | L8 |
| Membrane B | Test line | Verification line |
| Negative | L1 | L8 |

The results above validate this embodiment of the device of the invention.

1. A device for performing a test to determine the presence or absence of at least one analyte in a liquid sample comprising:
a) a support,
b) a porous matrix, fixed on the support, which allows the migration of the liquid sample, said matrix comprising:
(i) a liquid sample application area,
(ii) a labelling area, comprising at least one first binding partner bound to labelled particles, said first binding partner being capable of binding to said at least one analyte, if it is present in the liquid sample, and
(iii) at least one reaction area comprising:
a test results display area comprising at least one immobilised second binding partner capable of binding to said at least one analyte, and
a verification area which makes it possible to monitor the proper operation of the device downstream of the test results display area,
said liquid sample application area, labelling area and reaction area being in fluid communication;
wherein at the verification area, at least a part of the porous matrix has pores with dimensions smaller than those of the labelled particles, such that at least a proportion of the labelled particles from the labelling area is stopped at said verification area forming a symbol which is visible or displayable for a user.

2. The device according to claim 1, wherein the first binding partner and the second binding partner are chosen from the group consisting of antibody, antibody mixture, antibody fragment, mixture of antibody fragments, nanofitin, mixture of nanofitins, antigen, mixture of antigens, protein, mixture of proteins, polypeptide, mixture of polypeptides, peptide, mixture of peptides.

3. The device according to claim 1, wherein the labelled particles comprise an elastomer material, preferably latex.

4. The device according to claim 1, wherein the particles of labelled comprise colloidal gold.

5. The device according to claim 1, wherein the symbol is a straight line which is perpendicular to the main flow direction of the liquid sample.

6. The device according to claim 1, wherein the symbol forms a trace admitting several distinct tangents, in particular a curvilinear trace, or a broken-line trace, for example a zig-zag.

7. The device according to claim 1, wherein the verification area comprises a groove arranged in one of the faces of the porous matrix.

8. The device according to claim 7, wherein the groove results from plastic deformation of the porous matrix.

9. The device according to claim 8, wherein the groove results from thermal deformation of the porous matrix, for example by means of a heating blade or a laser.

10. A device according to claim 6 wherein a cross-section of the groove is in the shape of a “U”, a “V” or a rectangle.

11. The device according to claim 1, wherein the support is made from a liquid-impermeable material, preferably a synthetic plastic material.

12. The device according to claim 1, comprising a cassette placed around the matrix, the cassette having a first aperture arranged to allow access to the sample application area and at least a second aperture arranged to allow a user to see the reaction area.

13. A method for manufacturing a device according claim 1, the method comprising the steps:
producing a device according to the preamble of claim 1;
modifying the porous matrix, such that part of said matrix has, at the verification area, pores with dimensions smaller than those of the labelled particles.

14. The method according to claim 13, wherein the modification to the matrix is made by plastic deformation of the matrix by exerting localised pressure, preferably by rolling at least one rotating member bearing on the external face of the matrix.

15. The method according to claim 14, wherein the rotating member has a rolling diameter of at least 1 mm, preferably a rolling diameter of between 1 and 150 mm.

16. The method according to claim 13, wherein the modification of the matrix is made by plastic deformation of the matrix by exerting localised frontal pressure, preferably by applying a pressing member bearing on the external face of the matrix.

17. The method according to claim 16, wherein the pressing member has a diameter or an external width of between 0.1 mm and 4 mm.

18. The method according to claim 13, wherein the localised pressure is exerted by a crushing force of between 5N and 50N, preferably between 10N and 40N.
